(19) United States
(12) Patent Application Publication
(10) Pub. No.: US 2019/0053817 A1
(43) Pub. Date: Feb. 21, 2019

(54) CIRCUMFERENTIAL IRRIGATION METHOD OF SINUSITIS TREATMENT

(71) Applicant: Ondine International Holdings Ltd., St. Michael (BB)

(72) Inventors: Nicolas Gaston Loebel, Woodinville, WA (US); Tom Eugene Dawson, Bothell, WA (US); David Matthew Meller, Seattle, WA (US); Saran Kumar Baskaran, Mill Creek, WA (US); Michael Wefers, Granite Bay, CA (US)

(21) Appl. No.: 16/100,383

(22) Filed: Aug. 10, 2018

Related U.S. Application Data
(60) Provisional application No. 62/546,854, filed on Aug. 17, 2017.

(51) Int. Cl. A61B 17/24; A61M 3/02

(52) U.S. Cl. CPC A61B 17/24; A61M 25/0054; A61M 3/0283

(57) ABSTRACT

The present invention provides a method to treat sinusitis comprising (a) inserting a portion of an irrigation catheter having an expandable material in a non-expanded state endoscopically into a sinus of a patient through ostium of a sinus; (b) expand the expandable material under endoscopic visualization until it has expanded and filled some portion of the sinus without directly contacting and compressing mucosal surfaces of the sinus; (c) introducing irrigation fluid into the sinus through an irrigation lumen of the catheter to create a circumferential irrigation flow in space between the expanded material and the mucosal surface in order to dislodge and express sinus contents.

### CLAIM OF BENEFIT OF FILING DATE

This application claims the benefit of the filing date of U.S. Provisional Application Ser. No. 62/546,854 titled: “CIRCUMFERENTIAL IRRIGATION METHOD OF SINUSITIS TREATMENT” filed on Aug. 17, 2018, which is incorporated herein by reference for all purposes.

### FIELD OF INVENTION

The present invention relates to a method of treatment of sinusitis, especially chronic recurrent sinusitis using an irrigating, self-expandable catheter to express/expel materials from the paranasal sinuses.

### BACKGROUND

Sinusitis is an inflammatory disease of the paranasal sinuses. The paranasal sinuses are connected hollow cavities in the skull. Referring to FIGS. 1-2, these hollow cavities include the following: the maxillary sinus(es) 2, the ethmoid sinus(es) 4, the frontal sinus(es) 6, and the sphenoid sinus(es) 8. The maxillary sinus 2 is located inferior to the eye orbit 10 and lateral to the nasal cavity 12. The ethmoid sinus 4 is located superior to the maxillary sinus 2. The frontal sinus 6 is located superior to the ethmoid sinus 4. The sphenoid sinus 8 is located posterior to the ethmoid sinus and superior to the maxillary sinus 2. The sinuses (2, 4, 6, 8) contain cells that produce mucus, which is transported by mucociliary transport from the sinuses (2, 4, 6, 8) through their openings, known as sinus ostia, to the nasal cavity 12.

Chronic Recurring Sinusitis (“CRS”) is persistent inflammation of the sinus and nasal passages that is defined as lasting longer than 12 weeks or occurring more than 4 times per year with symptoms usually lasting more than 20 days. In clinical practice, there is a significant subpopulation of patients with CRS who remain resistant to cure despite rigorous treatment regimens including surgery, allergy therapy and prolonged antibiotic therapy. The reason for treatment failure is thought to be related to the destruction of the sinus mucociliary defense by bacteria that are not cleared from the sinuses.

The use of balloon catheters to treat sinus diseases has been disclosed. For example, U.S. Pat. No. 8,114,113 disclosed the use of a balloon catheter to dilate an ostium or duct, or the choana, to create a new opening (ostium) from a sinus to the nasal cavity, and to conduct sinusotomy. These methods often cause damage to the mucosal tissue and trauma to the underlying bone and treatment area.

### BRIEF DESCRIPTION OF THE DRAWINGS

FIG. 1 is a schematic front view of a portion of a human face showing the paranasal sinuses and nasal cavity;

FIG. 2 is a schematic lateral cross-sectional view of the paranasal sinuses;

FIG. 3 is a diagrammatic perspective view of a balloon catheter that can be used in accordance to a method of the present invention;

FIG. 4 is a close-up diagrammatic axially cross-sectional view of a portion of the balloon catheter;

FIG. 5 is a close-up diagrammatic longitudinally cross-sectional view of a portion the balloon catheter with its deflated balloon;

FIG. 6 is a schematic view of circumferential irrigation in a maxillary sinus in accordance to a method of the present invention;

FIG. 7 is a diagrammatic longitudinally cross-sectional view of another embodiment of a balloon catheter that can be used in accordance to a method of the present invention; and

FIG. 8 is a diagrammatic axially cross-sectional view of a portion of the balloon catheter shown in FIG. 7.

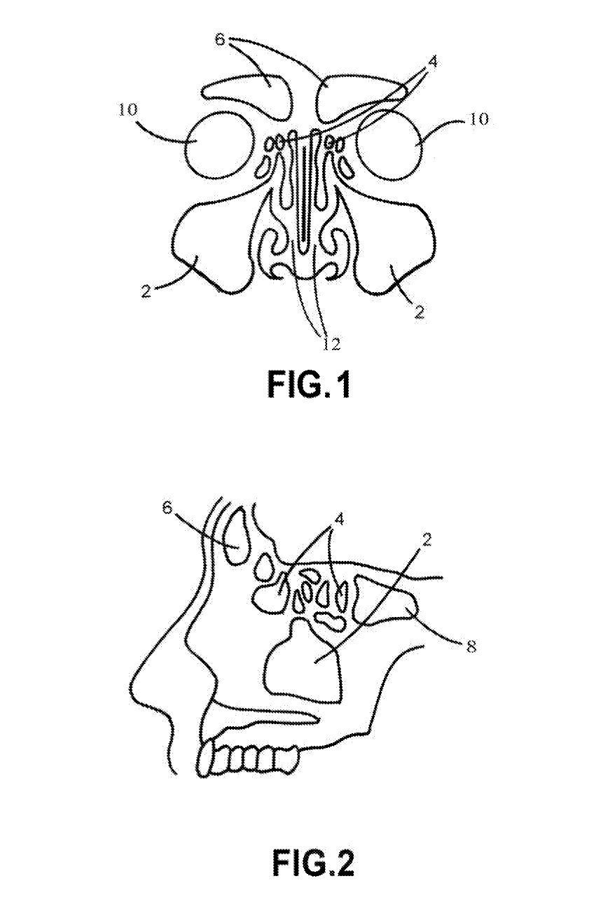

### DETAILED DESCRIPTION OF THE INVENTION

The present invention provides a method to treat sinusitis, especially CRS, that reduces or even prevents tissue damage and trauma to the treatment area by using an irrigating self-expandable catheter to fill the majority of the unoccupied volume of a sinus cavity and to create an irrigating fluid flow circumferentially around its expanded volume in order to improve the evacuation of materials residing on the surface of the sinus mucosa of the sinus cavity. The present invention has successfully treated sinusitis including acute sinusitis and CRS.

This application is related to and claims the benefit of the filing date of U.S. Provisional Application Ser. No. 62/546,854, filed Aug. 17, 2017, the contents of that application being hereby incorporated by reference herein for all purposes.

The irrigating self-expandable catheter used for the present invention can be any art-disclosed device that not only provides irrigation but also can enter a sinus cavity and then can expand into a desired volume so as to create an irrigating fluid flow circumferentially around the expanded volume

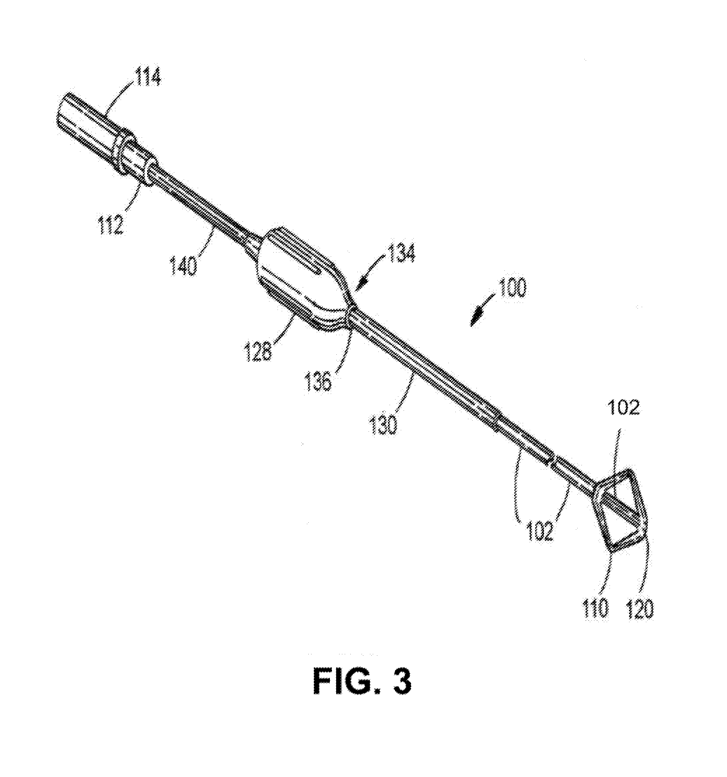

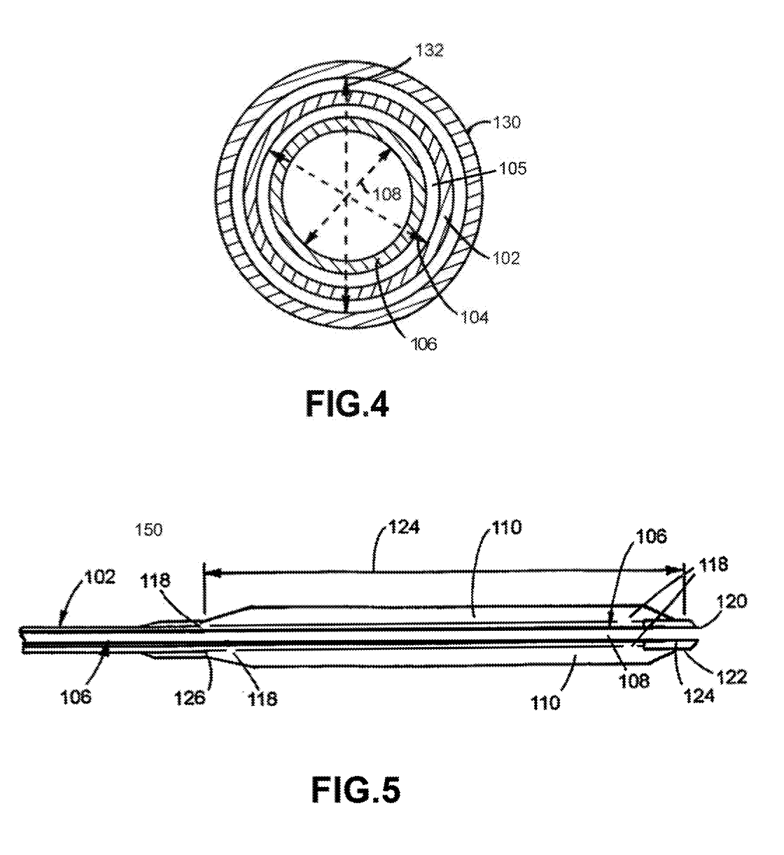

In one exemplary embodiment and referring to FIGS. 3-5, the self-expandable catheter is a balloon catheter 100 having an outer tube 102 with an outer lumen 104 and an inner tube 106 with an inner lumen for irrigation (hereinafter shall be referred to as irrigation lumen) 108, an inflatable balloon 110, and a fluid connector 112. At least a portion of the inner tube 106 is located within the outer lumen 104. The tubes (102, 106) and the lumens (104, 108) are coaxial thereby creating a lumen 105 in the space between the outer tube 102 and the inner tube 106 as shown in FIG. 4. This lumen 105 is used to inflate the balloon 110 with fluid and hereinafter shall be referred to as “balloon lumen” 105). The outer tube 102 and the inner tube 106 are constructed of flexible materials so that they can flex, bend, and conform to the passage ways of the nasal cavity, ostia, and sinus(es). The outer tube 102 also provides structural support to the catheter 100. The outer tube 102 can be constructed of any art-disclosed material suitable to provide support and flexibility such as aluminum alloy, titanium alloy, or the like. The inner tube 106 is constructed of any art-disclosed materials suitable to provide flexibility and conformability such as plastic tubing, polyurethane tubing, silicone tubing, Tygon® tubing, or the like.

The connector 112 is located at proximate end of the catheter 100. The proximate end of the connector connects the catheter 100 to a fluid source 114. In one embodiment, the fluid source 114 provides separate reservoirs in order to separate the fluid going into the catheter 100 and the fluid coming out of the catheter 100. For example, the reservoir for inflating of the balloon 110 is sized to properly inflate the balloon 110 without over-inflation (e.g., approximately 20 mL). The volume of the reservoir for irrigation can vary considerably (e.g., approximately 60 mL) based on irrigation needs and user preference. The connector 112 can include any suitable art-disclosed connector or connectors (e.g., luer lock(s), ferrule(s), or the like).

Referring to FIG. 5, the balloon 110 is attached to the distal region 116 of the catheter 100. The outer tube 102 contained within the distal region 116 contain port(s) 118 that transport fluid in and out of the balloon 110 during its inflation and deflation processes. In one embodiment, a capped means 120 is connected to the distal end 122 of the balloon 110 and the distal end 124 of the outer tube 102. The inner tube 106 is contained within the end cap means 120 and terminates that the distal end of the end cap means thereby allowing the irrigation lumen 108 to dispense irrigation fluid 146 out of the catheter 100.

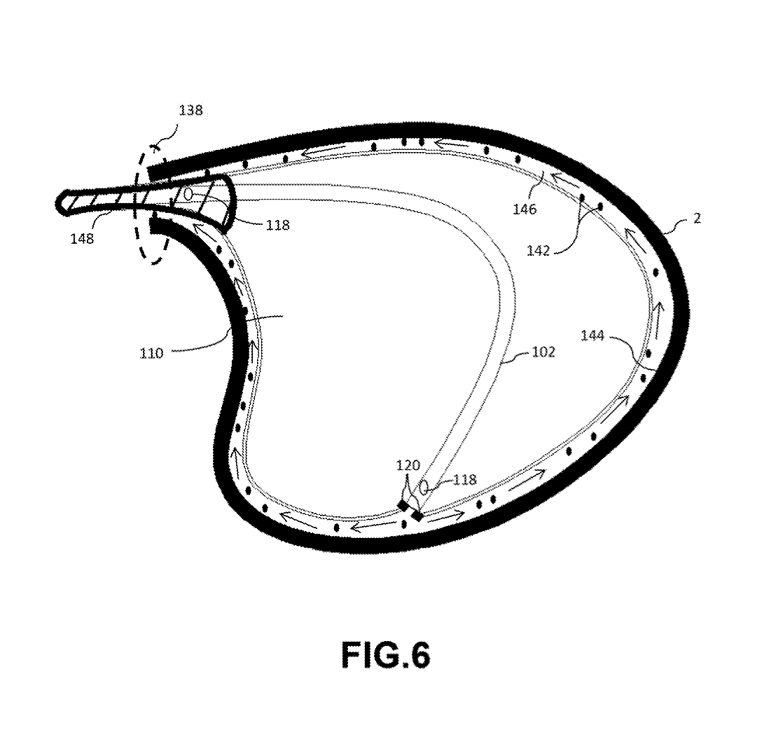

Referring to FIG. 6, the balloon 110 includes an optional shape restriction feature 148 in order to further ensure the balloon 110 is inflated by the port(s) 118 in a fashion that it ensure certain area(s) in or near the sinus (e.g., ostium 138 or the like) is not compressed by the inflated balloon 110. The shape restriction feature 148 can also prevent the inflated balloon 110 from expanding or bulging backwards in a retrograde fashion out of the ostium 138 of the sinus 2.

During operation, the balloon 110 is in fluid communication with the balloon lumen 105. The balloon 110 is constructed of resilient biocompatible material such as urethane, polyethylene terephthalate, or the like. Depending on the size and shape of the sinus(es) designated for treatment (“sinus treatment area”), the balloon 110 can be any size and shape suitable for the sinus treatment area. For example, the balloon 110 can be generally ellipsoidal shape, spherical with taped ends, cylindrical with taped ends, or the like. The diameter, length, and total volume of the inflated balloon 110 depend upon the anatomy of the patent's paranasal sinuses. For example, the diameter of the inflated balloon 110 may range from about 2 mm to about 10 mm, and from about 3 mm to about 9 mm or the like. The length of the inflated balloon may range from about 5 mm to about 50 mm, from about 9 mm to about 40 mm, and from about 20 to about 30 mm. The deflated balloon 110 has a size and shape that can gain access to the treatment site.

Referring to FIGS. 3-5, the catheter 100 further includes a fluid control assembly 128, a support tube 130 having a support lumen 132. Referring to FIG. 4, which shows a close-up cross-sectional view of the catheter 100 at a location near the distal end 134 (see FIG. 3) of the fluid control assembly 128, a portion of the outer tube 102 containing a portion of the inner tube 106 are contained within the support lumen 132. The outer tube 102, the inner tube 106 and the support tube 130 are all coaxial. The support tube 130 provides stiffness to the catheter 100. The support tube 130 can be constructed of any art-disclosed material suitable to provide the desired stiffness to the catheter 100 such as stainless steel, other metal alloys, or the like.

Referring to FIG. 3, the proximate end of the outer tube 102, the proximate end of the inner tube 106, and the proximate end 136 of the support tube 130 are all connected to the fluid control assembly 128 and a protective tube 140 with a protective lumen (not shown) serves as the conduit for fluid communication between the connector 112 and the fluid control assembly 128.

The fluid control assembly 128 uses art-disclosed fluid control means to control (i) the fluid communication between the fluid source 114 and the balloon lumen 105, and (ii) the fluid communication between the fluid source 114 and the irrigation lumen 108. Furthermore, the fluid control assembly 128 also serves as a handle for a user to hold and control the catheter 100.

During operation, the fluid control assembly 128 allows the fluid source 114 to be in fluid communication with the irrigation lumen 108 and/or the balloon lumen 105. When the connector 112 is connected to the fluid source 114, the fluid control assembly 128 allows the irrigation lumen 108 and/or the balloon lumen 105 to be in fluid communication with the fluid source 114 and can deliver (i) fluid from the fluid source 114 to the balloon 110 via the balloon lumen 105 for inflation of the balloon 110; (ii) from the balloon 110 to the fluid source 114 for deflation of the balloon 110; (iii) fluid from the fluid source 114 to the irrigation lumen 108 for dispensing fluid out of the catheter 100 in order to create an circumferential irrigating fluid flow around the inflated balloon 110.

Referring to FIG. 6, the inflated balloon 110 is not in direct contact with the mucosal surface 144 of the treated sinus 2; instead the inflated balloon 110 redirects the flow of irrigation fluid 146 dispensed from the irrigation lumen 108 around the exterior of the inflated balloon 110 between the inflated balloon's 110 exterior surface and the mucosal surface 144. This method is hereinafter referred to as “circumferential irrigation” method.

Unlike the circumferential irrigation method described above, the fluid exiting the end of a conventional irrigation catheter is not constrained and travels in a direction approximately normal (i.e., generally perpendicular) direction to the mucosal surface 144. Furthermore, even in the case of an irrigation catheter in which the irrigation fluid exits through multiple side holes and is directed in multiple directions towards the mucosal surface 144, the resulting fluid stream will impact the mucosal surface 144 only in a generally perpendicular direction.

The circumferential irrigation method uses the combination of the inflated balloon 110 and irrigation fluid 146 dispensed from the catheter 100 under pressure to optimally channel the flow of irrigation fluid 146 tangentially (e.g., not in a direct perpendicular direction) along the mucosal surface 144 creating a tangential or circumferential irrigation flow that is atraumatic to mucosal surface 144 but more effectively dislodges and expresses the sinus contents 142 (e.g., debris, biofilms, mucus, etc.) from the mucosal surface 144. Compared to conventional irrigation, the present invention's method further improves lumenal expression of the sinus contents 142 by more effectively detaching the sinus contents 142 from the mucosa surface 144 and pushing the resulting material through the sinus ostium 138 and out of the sinus cavity.

The present invention also contemplates that to deflate the balloon 100, fluid contained within the balloon 110 can be alternatively transported from the balloon 110 out of the catheter 100, via the inner lumen 108, the fluid control assembly 128 and the connector 112, into other disposal means (i.e., not the fluid source 114).

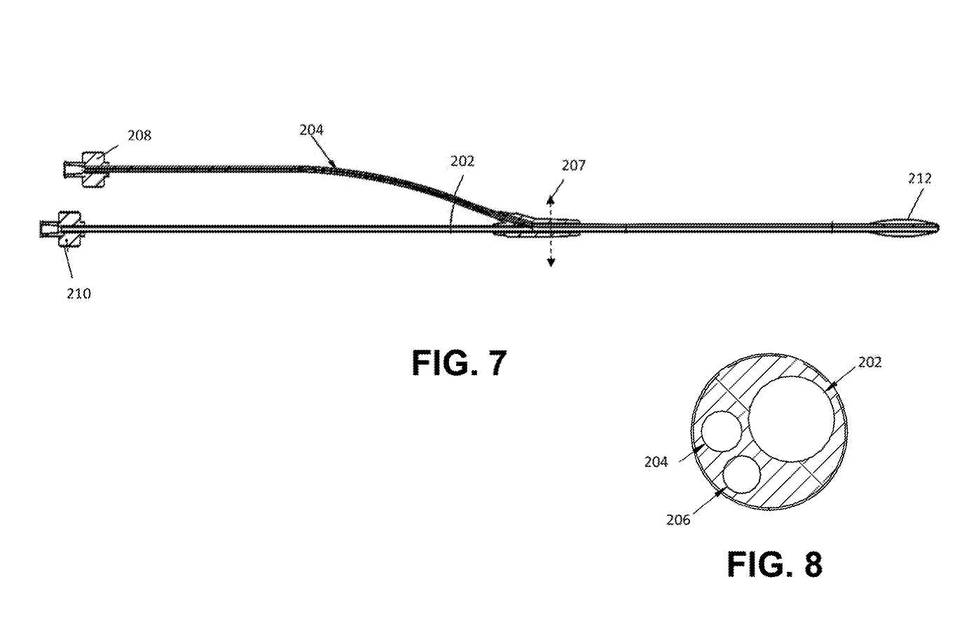

Referring to FIG. 7-8, an alternative embodiment of the balloon catheter 200 is presented with non-coaxial lumens 202, 204, 206. FIG. 8 shows the axially cross-sectional view of the balloon catheter 200 at 207 of the catheter handle 214 shown on FIG. 7. Non-coaxial lumen 206 provides access for a guide wire. Non-coaxial lumen 204 is connected to the fluid connector 208 at its proximate end and to the balloon 212 at its distal end. Non-coaxial lumen 204 functions same as the balloon lumen 105 described above. Non-coaxial lumen 202 is connected to the fluid connector 210 at its proximate end. During operation when the balloon 212 is inflated, the non-coaxial irrigation lumen 202 is in fluid communication with the fluid source 114 via the fluid connector 210 allowing the non-coaxial lumen 202 at its distal end to provide the irrigation fluid 146 necessary for circumferential irrigation of the mucosa surface 144. Non-coaxial irrigation lumen 202 functions same as the irrigation lumen 108 described above.

Other art-disclosed balloon catheters may be used in accordance to the present invention, including but not limited to, the balloon catheters disclosed in U.S. Pat. No. 8,114,113. In addition to balloon catheters, other art-disclosed self-expandable catheters may also be used in accordance to the present invention such as the expandable nasal stent (using expandable foam) as described in U.S. Pat. No. 5,336,163 or the like. To avoid unnecessary trauma to the patient, it may be desired that the diameter of the catheter 100 that enters the nasal cavity and into the any of the sinuses is sized sufficiently small in order to fit into the nasal passages including ostia.

To treat sinusitis, including acute sinusitis and CRS, the clinician normally identifies a particular sinus of interest to treat via CT, endoscopic evaluation and patient report. Once the sinus of interest has been identified, the clinician may optionally open the ostium to such sinus via a standard uncinectomy and antrostomy procedure, and the patient allowed to heal. Alternatively, this option step of opening the ostium may be omitted and the clinician proceeds directly to the method of the present invention.

The method of the present invention includes the first step of inserting a portion of the catheter (100, 200) having the balloon (110, 212) or another expandable material in a deflated state endoscopically into a sinus (e.g., the maxillary sinus 2, the ethmoid sinus 4, the frontal sinus 6, or the sphenoid sinus) through the sinus' ostium (such as the maxillary ostium 138 shown on FIG. 6). The catheter (100, 200) is connected to the fluid source 114 via the connector(s) (112, 208, 210). The inserted portion of the catheter (100, 200) is sufficiently flexible but with sufficient bending stiffness to allow the clinician to appropriately bend it to allow for easy insertion into the sinus. In another embodiment, the appropriate bending stiffness is provided by guiding wire placed within the non-coaxial lumen 206. Bending of the inserted portion of the catheter (100, 200) is performed prior to endoscopic insertion into the sinus. It is optional that a soft, thin, flexible guidewire is first endoscopically placed into the sinus using conventional methods. The inserted portion of the catheter (100, 200) is then placed into the desired location within the sinus by being attached to (e.g., threaded over or the like) and advanced over the guidewire. It is optional to rinse the sinus' ostium using conventional means prior to insertion of the catheter (100, 200) or just use the catheter (100, 200) to remove as much dislodgeable contents as possible from the ostium 138.

Once the balloon (110, 212) is placed into the sinus 2 endoscopically, the second step of the present invention includes inflating the balloon (110, 212) with fluid from the fluid source 114 via the port(s) 118 using the fluid control assembly 128 or via the connector 208 and the reservoir for the fluid source 114 (e.g., syringe or the like) under endoscopic visualization until the balloon (110, 212) inflates to fill a portion of the sinus 2, that portion being dependent on the sinus 2 being treated and the amount of sinus contents 142. If using an expandable material other than the balloon (110, 212), the expandable material shall be expanded at the desired location to fill a portion of the sinus 2. The method does not require the balloon (110, 212) or the other type of expandable material to exert direct pressure on the mucosal surfaces 144 of the sinus 2 (e.g., fill up the entire space or cavity within the sinus 2. The method also does not require the balloon (110, 212) or the other type of expandable material to physically compress the mucosal surface 144. Furthermore, the balloon (110. 212) or the other type of expandable material should not inflate/expand to a point that it occludes the sinus ostium 138.

Referring to FIG. 6, the inflated balloon (110, 212) fills some portion of the sinus 2 without directly contacting and compressing the mucosal surfaces 144. Neither the sinus 2 nor its associated ostium 138 itself is dilated to any appreciable degree. The third step of the present invention includes dispensing irrigation fluid 146 from the catheter 100 via the irrigation lumen using the fluid control assembly 128 or the connector 210. The fluid control assembly 128, or the connector 210 and its associated reservoir for the fluid source 114, controls the amount of fluid 146 coming from the fluid source 114 into the irrigation lumen (108, 202). The fluid 146 is introduced under pressure through the irrigation lumen (108, 202) into the sinus 2 and is directed around the circumference of the inflated balloon (110, 212) or the other type of expandable material in the space between it (i.e., inflated balloon/expandable material) and the mucosal surface 144.

Once the clinician feels that the treated sinus 2 has been sufficiently cleared of its sinus contents 142, the fourth step of the present invention includes deflating the balloon (110, 212) by transporting the fluid contained within the balloon 110 back out of the catheter 100 to either the fluid source 114 or other disposal means. If a non-balloon expandable material is used, then other art-disclosed methods of deflation may be used. The fifth and final step of the present invention includes removing the inserted catheter 100 from the patient.

The method of the present invention set forth above can be repeated to treat the remaining affected paranasal sinuses.

The particulars shown herein are by way of example and for purposes of illustrative discussion of the invention only and are presented in the cause of providing what is believed to be the most useful and readily understood description of the principles and conceptual aspects of the invention. In this regard, no attempt is made to show structural details of the invention in more detail than is necessary for the fundamental understanding of the invention, the description taken with the drawings making apparent to those skilled in the art how the several forms of the invention may be embodied in practice. It is understood that the present invention as described and claimed herein can be used for many additional purposes, therefore the invention is within the scope of other fields and uses and not so limited. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A method to treat sinusitis comprising:
(a) inserting a portion of an irrigation catheter having an expandable material in a non-expanded state endoscopically into a sinus of a patient through ostium of a sinus;
(b) expanding the material of the catheter under endoscopic visualization until the material expands and fills some portion of the sinus without directly contacting and compressing mucosal surfaces of the sinus; and
(c) introducing irrigation fluid into the sinus through an irrigation lumen of the catheter to create a circumferential irrigation flow in space between the expanded material and the mucosal surface in order to dislodge and express sinus contents.

2. The method of claim 1 wherein:
(a) the expandable material is a balloon;
(b) expanding the material step is performed by inflating the balloon with fluid from a fluid source;
(c) the method further comprising:
(i) deflating the balloon by transporting the fluid contained within the balloon back out of the catheter; and
(ii) removing the catheter from the patient, wherein the ostium is not occluded by the method.

3. The method of claim 1 wherein the expandable material is an expandable foam.

4. The method of claim 1 wherein the irrigation catheter includes a balloon and a balloon lumen for inflating and deflating the balloon during operation.

5. The method of claim 1 wherein the irrigation catheter includes a shape restriction feature.

6. The method of claim 1 wherein the irrigation fluid is dispensed by the catheter under pressure and the expanded material channels flow of the irrigation fluid tangentially along the mucosal surface creating the circumferential irrigation flow.

7. The method of claim 1 wherein the method including the circumferential irrigation flow is atraumatic to the mucosal surface.

8. The method of claim 1 wherein the irrigation catheter includes coaxial lumens.

9. The method of claim 1 wherein the irrigation catheter includes non-coaxial lumens.

10. The method of claim 1 wherein the irrigation catheter includes a fluid control assembly.

11. The method of claim 1 wherein the irrigation fluid originates from a reservoir distinct and separate from the reservoir containing the fluid to inflate the balloon.

12. The method of claim 1 wherein the reservoir containing the fluid source for inflation of the balloon is a syringe.

13. A method to treat sinusitis comprising:
(a) inserting a portion of an irrigation catheter having a balloon in a deflated state endoscopically into a sinus of a patient through ostium of a sinus;
(b) inflating the balloon of the catheter with fluid from a fluid source under endoscopic visualization until the balloon inflates and fills some portion of the sinus without directly contacting and compressing mucosal surfaces of the sinus;
(c) introducing irrigation fluid into the sinus through an irrigation lumen of the catheter to create a circumferential irrigation flow in space between the inflated balloon and the mucosal surface in order to dislodge and express sinus contents;
(d) deflating the balloon by transporting the fluid contained within the balloon back out of the catheter; and
(e) removing the catheter from the patient, wherein the ostium is not occluded by the method.

14. The method of claim 13 wherein the method including the circumferential irrigation flow is atraumatic to the mucosal surface.

15. The method of claim 13 wherein the irrigation catheter includes a shape restriction feature.

16. The method of claim 13 wherein the irrigation fluid is dispensed by the catheter under pressure and the inflated balloon channels flow of the irrigation fluid tangentially along the mucosal surface creating the circumferential irrigation flow.

17. The method of claim 13 wherein the irrigation catheter includes a fluid control assembly.

18. The method of claim 13 wherein the irrigation fluid originates from a reservoir distinct and separate from the reservoir containing the fluid to inflate the balloon.

19. The method of claim 13 wherein the reservoir containing the fluid source for inflation of the balloon is a syringe.

20. The method of claim 13 wherein the irrigation catheter includes non-coaxial lumens.
